(19) United States
(12) Patent Application Publication
(10) Pub. No.: US 2011/0313383 A1
(43) Pub. Date: Dec. 22, 2011

(54) WOUND DRESSING

(76) Inventors: Juergen Hofstetter, Heidenheim (DE); Albert Manes, Barcelona (ES)

(21) Appl. No.: 12/998,936

(22) PCT Filed: Dec. 21, 2009
(86) PCT No.: PCT/EP2009/009190

§ 371 Date: Aug. 31, 2011

(30) Foreign Application Priority Data
Dec. 23, 2008 (DE) 10 2008 062 824.7

(51) Int. Cl. A61L 15/44

(52) U.S. Cl. 604/372; 604/367

(57) ABSTRACT

The present invention relates to a wound dressing, comprising a carrier layer and an absorbing cushion. The absorbing cushion comprises at least two different absorbing layers that are separated by a third layer. The third layer comprises at least one antimicrobially acting agent that exhibits the effect thereof within the absorbing cushion.

The present invention relates to wound dressings and their use in modern wound treatment. More particularly, the present invention relates to wound dressings comprising two mutually different absorbent layers, especially two differently capable absorbent layers, for treating highly or moderately exudating wounds.

A multiplicity of products have been put on the market in the past in connection with the healing of wounds, especially wounds caused by operative interventions. In addition, a multiplicity of proposals as to how and with what a wound can be made to heal are discernible from the patent literature as well as the trade literature.

The German application DE-19727032-A1, for instance, describes a wound bandage for postoperative use, including two differently capable bonding adhesive regions surrounding an absorbent pad. Fibrous nonwoven webs in particular can be used here as absorbent pads. The differently capable bonding adhesive regions provide for easier removal from the skin in that there is no damaging of predamaged skin.

The two U.S. Pat. No. 5,465,735-A and U.S. Pat. No. 5,632,731-A describe wound dressings which do not stick to the wound, these wound dressings comprising a backing layer and an absorbent pad. The absorbent pads have a multi-ply construction in that each pad comprises two different fibrous nonwoven webs of differing fiber density which are covered on the wound side by a perforated foil that is non-adherent to the wound. The use of a film which is non-adherent to the wound means that removal of the dressing from the wound is not a traumatic experience for the patient.

The international application WO-98/41095-A1 describes a multi-ply antimicrobial wound dressing which includes a wound contact layer, an absorbent layer and a covering layer. The wound contact layer to be used here is a polymer foil with a silver coating wherein the silver is in atomic disorder.

It may additionally be provided here that further layers are likewise coated with silver. The use of silver in atomic disorder is said to provide an increased release of silver in the presence of electrolytes compared with silver without atomic disorder.

The international application WO-01/60599-A1 describes multilayered antimicrobial wound dressings comprising a wound contact layer formed of a silver-coated textile material such as, for example, a silver-coated woven or nonwoven fabric, which is coated with a gel. What is essential here is that silver is released by the wound dressing into the wound in the form of ions for use against germs in a wound.

The international application WO-2004/112852-A1 describes an antimicrobial wound dressing comprising a wound contact film coated with silver on one side. The silver-coated side faces an absorbent layer. This arrangement is said to provide an antimicrobial wound dressing which releases silver into the wound in an amount sufficient for germ killing without any discoloration occurring due to silver abrasion.

The German application DE-10108083-A1 describes an odor-adsorbing wound dressing containing silver as an antimicrobial agent. This antimicrobial agent is obligatorily present in or on a layer other than the odor-adsorbing layer. This is said to provide a wound dressing which has improved odor neutralization as well as being at the same time capable of releasing silver and thereby killing germs in a wound.

The wound dressings thus described all have the disadvantage that the wound dressings in their intended use release a very high concentration of silver into the wound.

The present invention accordingly has for its object to provide a wound dressing having improved absorbency properties which even in the event of prolonged use on a strongly or moderately exudating wound counteracts any contamination with germs without burdening the wound with excess active ingredients. It is a further object to provide a wound dressing which counteracts the maceration of the wound and of the surrounding areas of the skin. It is a further aspect to also provide a wound dressing which in the event of prolonged use reduces the risk of sticking to the wound and at the same time counteracts maceration.

These objects are achieved by a wound dressing according to the features of claim 1. Accordingly, a wound dressing according to the present invention comprises a backing layer and an absorbent pad, wherein the absorbent pad comprises a first absorbent layer comprising a first absorbent material and a second absorbent layer which is other than the first layer and comprises a second absorbent material other than the first material, wherein the first layer is distanced from the second layer by at least one third layer, and this third layer comprises at least one antimicrobial agent.

Absorbency in connection with the present invention is always and exclusively to be understood as meaning the imbibition of liquids. Furthermore, in connection with the present invention, distancing is to be understood as meaning the spatial separation of two components and that this spatial separation can have been effected completely or merely in parts; that is, the two distanced components can be distanced in subregions and in direct contact in further subregions.

It is thus possible to provide a wound dressing which comprises an antimicrobial agent which in use of the wound dressing does not come directly into contact with the wound, thereby not overloading the wound with antimicrobial agent. In addition, arranging the layers according to the present invention provides a wound dressing whose antimicrobial agent develops its effect almost exclusively within the absorbent pad. There is further provided a wound dressing which, depending on the wound exudate flux occurring, ensures that increased wound exudate is imbibed hence absorbed and kept away from the wound. This deprives any bacteria there of their breeding-ground.

A preferred embodiment provides in particular that the second absorbent layer is disposed between the first absorbent layer and the backing layer. Simultaneously or independently therefrom, it can be provided in this connection that the first absorbent layer is configured as a liquid distributor layer and the second absorbent layer as a liquid depot layer. However, both absorbent layers do have absorbent properties. More particularly, however, a wound dressing according to the present invention comprises first and second absorbent layers which comprise no antimicrobial agents before the intended use.

By way of absorbent materials, a multiplicity of materials known in wound management can be used according to the present invention. It will prove particularly advantageous to use fiber materials as absorbent materials for the first and/or second layer. It is provided more particularly in this connection to fabricate not only the first absorbent layer but also the second absorbent layer from fiber materials. It will further prove to be advantageous in this connection for the absorbent pad to comprise a first absorbent layer and a second absorbent layer wherein the second absorbent layer has a greater free absorbency than the first absorbent layer, wherein the free absorbency is determined according to DIN EN 13726-1 (2002). Simultaneously or independently therefrom, it is further advantageously provided in this connection that the first absorbent layer in the use state of the wound dressing faces the wound, wherein the first absorbent layer is further preferably in direct or indirect contact with the wound. When the wound dressing is used as intended, the wound exudate is accordingly kept away from the wound in that the first absorbent layer transports the wound exudate unidirectionally to the second layer and this second absorbent layer stores the wound exudate. It is thus also possible to provide a wound dressing which imbibes the wound exudate particularly efficiently to keep the wound surface very substantially dry and prevent any maceration. The unidirectional transportation of the wound exudate also ensures that the wound exudate comes into direct contact with the antimicrobial agent in the third layer, for the latter to develop their anti-microbial effect. The wound dressing can accordingly also remain on the wound for a prolonged period without any recontamination with germs due to the wound dressing.

It is also more particularly provided in this connection that the first absorbent layer has a free absorbency A₁ of at least 0.5 g/g, more particularly at least 2 g/g and even more preferably of at least 4 g/g, the free absorbency A₁ being determined in accordance with DIN-EN 13726-1 (2002). It is very particularly preferably provided in this connection that the first absorbent layer has a free absorbency A₁ of at most 10 g/g. It is very particularly preferable for the first absorbent layer to have a free absorbency A₁ of at least 1 g/g to 10 g/g, more particularly of at least 2 g/g to 10 g/g and even more preferably of at least 5 g/g to 10 g/g.

It is further preferably provided, simultaneously or independently therefrom, that the second absorbent layer has a free absorbency A₂ of at least 12 g/g, more particularly at least 15 g/g and even more preferably of at least 20 g/g, the free absorbency A₂ being determined according to DIN-EN 13726-1 (2002). It is very particularly provided in this connection that the second absorbent layer has a free absorbency A₂ of at most 50 g/g. It is very particularly preferable in this connection that the second absorbent layer has a free absorbency of at least 12 g/g to 50 g/g, more particularly of at least 15 g/g to 50 g/g and even more preferably of at least 20 g/g to 50 g/g.

Simultaneously or independently therefrom, a wound dressing according to the present invention includes an absorbent pad which has a free absorbency A_{T} of at least 8 g/g, more particularly of at least 12 g/g and even more preferably of at least 20 g/g, the free absorbency A_{T} being determined according to DIN-EN 13726-1 (2002). It is further preferably provided here that the absorbent pad has a free absorbency A_{T} of at most 50 g/g. It is very particularly preferable here that the absorbent pad has a free absorbency A_{T} of at least 8 g/g to 50 g/g, more particularly of at least 12 g/g to 50 g/g and even more preferably of at least 20 g/g to 50 g/g.

A wound dressing according to the present invention further includes, simultaneously or independently from each other, a first absorbent layer and a second absorbent layer which is other than the first absorbent layer, the free absorbencies A₁ and A₂ of which are such that the A₁:A₂ ratio corresponds to the ratio in the range from 1:2 to 1:10. The A₁ to A₂ ratio is more preferably equal to the ratio in the range from 1:3 to 1:10 and even more preferably in the range from 1:5 to 1:10.

A further embodiment of the present invention provides more particularly that the first absorbent material comprises cellulose fibers or viscose fibers. It may also be more particularly provided here that the first absorbent material comprises a fibrous nonwoven web comprising and more preferably consisting of viscose fibers and thermoplastic fibers, in which case it is further preferable that polypropylene or polyethylene fibers may be used as thermoplastic fibers. These thermoplastic fibers can also be present as so-called bicomponent fibers in the form of core-sheath fibers.

It is also more particularly provided that the second absorbent material comprises superabsorbent fibers. These fibers have a particularly high free absorbency and unlike particulate superabsorbent materials have the advantage that they are particularly convenient to commix with and hold in place by other fibers. Hence when superabsorbent fibers are used, there is no need for enveloping the second absorbent layer.

It is more particularly provided in this connection that the second absorbent layer comprises by way of absorbent material a fibrous nonwoven web comprising a blend of superabsorbent fibers and supporting fibers. However, it may also be provided here that the fibrous nonwoven web comprises superabsorbent fibers and supporting fibers and the supporting fibers are selected from the group of thermoplastic fibers and/or cellulose fibers and/or viscose fibers. It is very particularly preferred for the fiber blend to comprise superabsorbent fibers, viscose fibers and thermoplastic fibers wherein it is further preferable that polypropylene or polyethylene fibers can be used as thermoplastic fibers. These thermoplastic fibers can also be present as so-called bicomponent fibers in the form of core-sheath fibers.

The second absorbent material here preferably comprises a fiber blend comprising more than 20% by weight, preferably more than 30% by weight, more preferably more than 40% by weight, more preferably more than 50% by weight and even more preferably more than 60% by weight of superabsorbent fibers. This makes it possible to provide an absorbent pad having a particularly high free absorbency. A correspondingly endowed wound dressing can remain on the wound for up to three or more days.

The term “superabsorbent” refers to a water-swellable substantially water-insoluble material in the form of fibers which is capable of absorbing at least about 10 times, preferably about 20 times and more preferably about 50 times or more of its own weight of water. The superabsorbent material may be formed of an organic material which may comprise synthetic and/or natural material, for example agar, pectin and guar gum or else synthetic materials, for example synthetic hydrogel polymers. Synthetic hydrogel polymers comprise for example carboxymethylcellulose, alkali metal salts of polyacrylic acids, alkali metal salts of poly-methacrylic acids, polyacrylamides, polyvinyl alcohol, ethylene-maleicanhydride copolymers, polyvinyl ethers, hydroxypropylcellulose, polymers and copolymers of vinylsulfonic acid, polyacrylates, polymethacrylates, polyacrylate-polymethacrylate copolymers, poly-acrylamides and the like. Very particular preference for use as superabsorbent fibers is given to superabsorbent fibers formed from alkali metal salts of polyacrylic acids, alkali metal salts of poly-methacrylic acids, polyacrylates, polymethacrylates or polyacrylate-polymethacrylate copolymers. The hydrogel polymers are preferably lightly crosslinked to render the materials substantially water-insoluble. The crosslinking can be accomplished for example through irradiation or covalent, ionic, van der Waals or hydrogen bonding. Suitable materials are available from various manufacturers such as BASF and Stockhausen Inc.

It is further preferably provided here that the second absorbent layer comprises by way of absorbent material a fibrous nonwoven web comprising a blend of at least 20% by weight of superabsorbent fibers, at least 10% by weight of viscose or cellulose fibers and at least 5% by weight of thermoplastic fibers. It is very particularly preferable for the second absorbent layer to comprise by way of absorbent material a fibrous nonwoven web comprising a blend of at least 40% by weight of superabsorbent fibers, at least 20% by weight of viscose or cellulose fibers and at least 5% by weight of thermoplastic fibers. The presence of supporting fibers in the fibrous nonwoven web ensures that the liquid to be imbibed is uniformly distributed at the beginning of the absorbency.

Embodiments which will prove particularly advantageous further include wound dressings comprising an absorbent pad having a layer thickness S in the range from 0.1 to 10.0 mm. More particularly, a wound dressing according to the present invention includes an absorbent pad having a layer thickness S in the range from 0.1 to 8.0 mm, more particularly in the range from 0.1 to 7.0 mm and even more preferably in the range from 0.5 to 5.0 mm. Wound dressings having such layer thicknesses are not only capable of sustainably imbibing wound exudate but also of ensuring good drapability. These layer thicknesses can be the same at every point of the absorbent pad, or assume different values in different regions of the absorbent pad.

According to the present invention, the first and second absorbent layers are distanced by a third layer comprising an antimicrobial agent. This third layer thus provides an antimicrobial agent which develops its effect within the wound dressing and kills off germs within the wound dressing. It will be found particularly advantageous for the third layer to be relatively thin, compared with the first or second absorbent layer.

In a further development of the concept of the present invention, it will prove to be advantageous for the wound dressing to comprise by way of the third layer a polymer net, a perforated polymer foil, a fibrous nonwoven web or a textile material such as a knitted or woven fabric. It may also be more particularly provided in this connection that the third layer consists of a polymer net or a perforated polymer film which in either case includes a multiplicity of holes, openings or apertures or channels having an average diameter in the range from 0.01 mm to 1 mm and preferably in the range from 0.1 mm to 1 mm for passage of liquids, more particularly wound exudate.

It may further be preferable for the third layer to include a multiplicity of holes, openings, apertures or channels for passage of liquids, in which case these holes, openings, apertures or channels occupy an area of at least 20% of the area of the third layer. It is further preferably provided here that the holes, openings, apertures or channels occupy an area of at least 25%, more particularly at least 30% and even more preferably at least 35% of the area of the third layer. It is very particularly preferable for the wound contact layer to include holes, openings, apertures or channels which occupy an area of at least 20% and at most 80% of the area of the third layer. It is very particularly preferable for the wound dressing to include a wound contact layer including from 10 to 100 holes per cm². One possibility here is for the wound contact layer to be an apertured polymer film or a polymer net, more particularly of polyethylene or polypropylene. Thus, the first absorbent layer can be sufficiently distanced from the second absorbent layer while at the same time a sufficient amount of anti-microbial agents are provided within the wound dressing.

It is more particularly provided here that the anti-microbial agent has been applied or coated directly onto the third layer. However, it may also be provided that the third layer has been impregnated with the antimicrobial agent, or that the antimicrobial agent has been incorporated in the third layer.

It is particularly advantageous for a further embodiment to utilize by way of third layer a polymer net or a perforated polymer foil, a nonwoven or a textile material such as a knitted or woven fabric coated or impregnated with silver. However, it may also be provided that the silver is incorporated into the third layer. Simultaneously or independently therefrom, it may be more particularly provided in this connection that the silver is used in the form of elemental silver, silver oxide, silver salt or a silver complex or mixtures thereof. Hence a wound dressing according to the present invention comprises by way of anti-microbial agent more particularly elemental silver, silver oxide or silver in the form of silver complexes or silver salts. It is very particularly preferable to provide elemental silver as antimicrobial agent here. Silver in its elemental form in particular has a very low solubility product, meaning that, compared with other active antimicrobial ingredients, a very low concentration of active ingredient is released in the presence of wound exudate. Yet this concentration is sufficient to provide an antimicrobial effect within the wound dressing. Without wishing to be tied to the theory, it is believed that silver bound within a wound dressing according to the present invention develops its effect in elemental form at the level of the third layer and in the form of silver-complex ions or silver ions detached out of the elemental ensemble by the wound exudate. However, these ions tend to interact with oppositely charged ions. In the case of positively charged silver ions and the use of superabsorbent fibers composed of salts of polyacrylic acid or of polyacrylates, which are known to have negatively charged regions, it is even the silver-complex ions or silver ions released by the wound exudate which are detained by these fibers within the wound dressing. Thus, the silver can act within the wound dressing only except for a very low concentration, preventing any contamination with excessive amounts of silver on the wound.

In accordance with a more advanced concept, the absorbent pad releases less than 25 μg of silver per 1 g of absorbent pad over 24 hours at 25° C. in 100 ml of water or physiological saline, more particularly less than 15 μg of silver per 1 g of absorbent pad over 24 hours at 25° C. in 100 ml of water or physiological saline and more particularly less than less than 5 μg of silver per 1 g of absorbent pad over 24 hours at 25° C. in 100 ml of water or physiological saline with regard to silver in elemental form or in the form of salts or complexes. Atomic absorption spectroscopy is used to determine the amount of silver released.

In a further embodiment of the present invention, a wound dressing according to the present invention may comprise at least one or more further layers that perform various functions within the overall system of the wound dressing. It may be more particularly provided here that the absorbent pad further comprises a wound contact layer which is further preferably in immediate contact with the first or second layer. It is very particularly preferable here that this wound contact layer comprises no antimicrobial agents before the intended use of the wound dressing. Thus, it is possible to provide an antimicrobial wound dressing comprising an antimicrobial agent acting merely within the interior of the wound dressing.

In accordance with a further embodiment of the present invention it may also be provided that the wound dressing comprises a wound contact layer which has the same projected area as the backing layer. It may be provided here that the wound contact layer is in direct contact with the first or second layer in the region of the absorbent pad and is connected to the backing layer in the edge region of the wound dressing. In this embodiment, the wound contact layer combines with the backing layer to form an envelope for the absorbent pad.

It will further prove particularly advantageous here for the wound contact layer to be a polymer net or a perforated polymer foil. These polymer nets or perforated foils have the advantage that they do not stick to a wound. Thus, using such a foil or net also makes it possible to provide a wound dressing which does not stick to the wound.

It may also be particularly provided in this connection that the wound contact layer consists of a polymer net or a perforated polymer foil which in either case has a multiplicity of holes, openings or apertures or channels having an average diameter in the range from 0.01 mm to 1 mm and preferably in the range from 0.1 mm to 1 mm for passage of liquids, more particularly wound exudate.

It may further be preferable for the wound contact layer to include a multiplicity of holes, openings, apertures or channels for passage of liquids, in which case these holes, openings, apertures or channels occupy an area of at most 30% of the area of the wound contact layer. It is further preferably provided here that the holes, openings, apertures or channels occupy an area of at most 25%, more particular at most 20% and even more preferably at most 15% and very particularly preferably at most 10% of the area of the wound contact layer. It is very particularly preferable for the wound contact layer to include holes, openings, apertures or channels which occupy an area of at least 10% and at most 20% of the area of the first side of the wound contact layer. It is very particularly preferable for the wound dressing to include a wound contact layer including from 10 to 100 holes per cm². One possibility here is for the wound contact layer to be an apertured polymer film or a polymer net, more particularly of polyethylene or polypropylene.

In a further preferred embodiment of the invention, the wound dressing comprises by way of wound contact layer a polymer net or a perforated polymer foil composed of polyethylene or polypropylene, and a first absorbent layer having a first absorbent material which comprises a fibrous nonwoven web. This fibrous nonwoven web comprises from 80% to 99% by weight of viscose fibers and from 1% to 20% by weight of thermoplastic fibers, the thermoplastic fibers preferably being polypropylene or polyethylene fibers present as so-called bi-component fibers in the form of core-sheath fibers. It is particularly preferable for the fibrous nonwoven web in this embodiment of the invention to contain 85% by weight of viscose fibers and 15% by weight of bicomponent fibers in the form of core-sheath fibers. The proportion of bicomponent fibers in the fibrous nonwoven web can strengthen the adherence between the first absorbent layer and the wound contact layer.

According to the present invention, the backing layer may utilize any currently known backing material that has sufficient breaking strength and is very substantially impermeable to liquids. The backing layer of a wound dressing according to the present invention may utilize more particularly fibrous nonwoven webs or polymer films. It is further preferable for the backing layer of a wound dressing according to the present invention to more particularly utilize polymer films which are water impermeable and have a high water vapor permeability. Films fabricated from polyurethane, polyether urethane, polyester urethane, polyether-polyamide copolymers are particularly suitable. More particularly, a water impermeable and water vapor permeable polyurethane film, polyester urethane film or polyether urethane film is preferred for use as backing layer. Very particular preference is also given to polymer films from 15 to 50 μm, more particularly from to 40 μm and most preferably from 25 to 30 μm in thickness. The water vapor transmission rate of the polymer film of the wound dressing is preferably at least 750 g/m²/24 h, more particularly at least 1000 g/m²/24 h and most preferably at least 2000 g/m²/24 h (measured to DIN EN 13726-2). In particularly preferred embodiments, these films have a moistureproof tacky edge portion. This edge portion ensures that the wound dressing can be applied to and fixed at its intended location. Particularly preferable adhesives achieve in a thin add-on of 20 to 35 g/m² a water vapor transmission rate combined with the film of at least 800 g/m²/24 h and preferably of at least 1000 g/m²/24 h (measured to DIN EN 13726-2 (2002)).

In a further embodiment, however, it is also possible to use fibrous nonwoven webs as backing layer for a wound dressing according to the present invention. These fibrous nonwoven webs are more particularly fabricated from hydrophobic fibers and consolidated using jets of water.

However, it is also provided that a meltblown fibrous nonwoven web, spunlaced fibrous nonwoven web or airlaid fibrous nonwoven web is used as backing layer wherein these fibrous nonwoven webs are further preferably fabricated from polyurethane, polystyrene-isoprene block copolymers, polystyrene-butadiene block copolymers, polyesters, polyolefins or polyester phthalates.

The invention will now be elucidated with reference to a drawing without this drawing being intended to have any limiting effect with regard to the invention. In the drawing

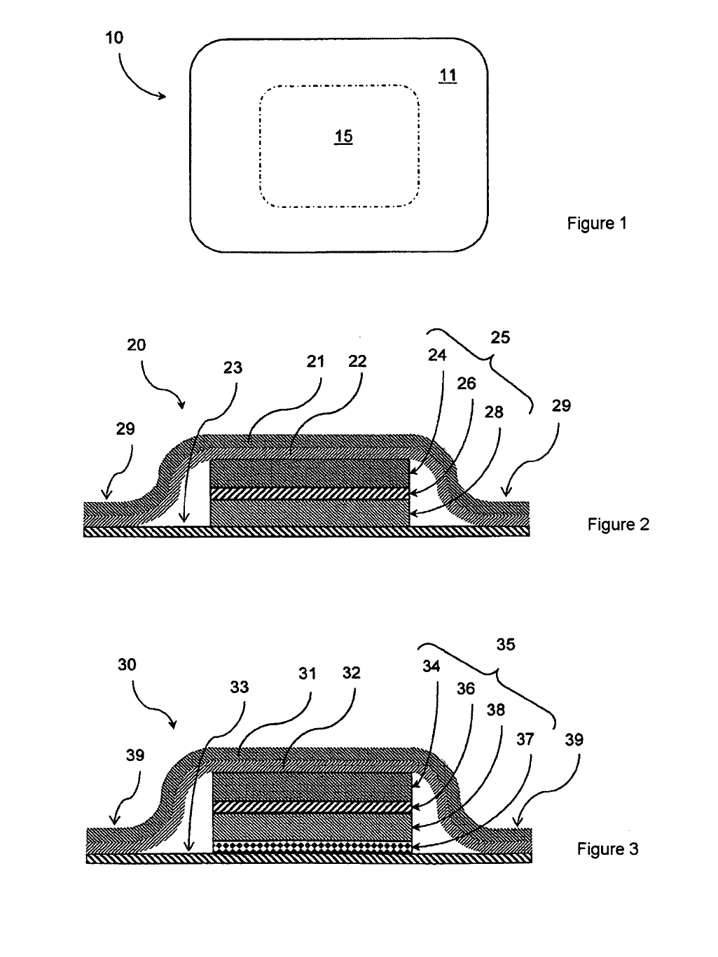

FIG. 1 shows an embodiment of an inventive wound dressing in plan view,

FIGS. 2 and 3 each show alternative embodiments of inventive wound dressings in cross section.

FIG. 1 shows a first inventive wound dressing 10 in plan view. The wound dressing includes a backing layer and an absorbent pad 15. The backing layer 11 is uniformly coated on one side, the reverse side, with a skin-friendly pressure-sensitive adhesive (not depicted). The absorbent pad 15 is disposed in the center of the backing layer so that a tacky margin for securement to the skin of a patient surrounds the absorbent pad on all sides. The wound dressing 10 is fabricated as a so-called island dressing.

FIG. 2 shows an inventive wound dressing 20 in cross section. The wound dressing includes a backing layer 21 composed of a water-repellent fibrous nonwoven web uniformly coated with an acrylate pressure-sensitive adhesive 22. In the center of the backing layer 21 the pressure-sensitive adhesive 22 has been used to apply an absorbent pad 25 leaving the absorbent pad surrounded on all sides by an uncovered tacky margin for fixing the wound dressing on the skin of a patient. The absorbent pad 25 is in a multilayered embodiment and consists of a first absorbent layer 28 composed of a first absorbent fiber material, a second absorbent layer 24 composed of a second absorbent material (a hydrophilic polyurethane foam) and a third layer 26 which is disposed between the first and second layers and distances the first and second layers. The third layer 26 consists of a netlike polyethylene foil coated with elemental silver on both sides. The polymer foil is coated such that the openings of the netlike foil remain clear for wound exudate passage, so that exudate can flow from the first absorbent layer to the second absorbent layer. The first absorbent layer 28 has a lower free absorbency than the second absorbent layer 24, so that wound exudate arising in the use state of the wound dressing is imbibed by the first absorbent layer in order ultimately to be stored by the second absorbent layer. In this way, the wound exudate is substantially kept away from the wound and any maceration prevented. The disposition of the silver-coated polymer foil between the two absorbent layers keeps the wound dressing very substantially germ-free—even in the event of a prolonged dwell time of three to five days on the wound—to prevent any recontamination of the wound with germs. The side of the wound dressing which in its use state faces the body of the patient is covered with a detachable release paper 23 for protection before use.

FIG. 3 shows a further inventive wound dressing 30 in cross section. The wound dressing consists of a backing layer 31 composed of a water-repellent fibrous nonwoven web uniformly coated with a pressure-sensitive adhesive 32. The laminate consisting of the pressure-sensitive adhesive 32 and the backing layer 31 is permeable to water vapor to provide a breathable wound dressing. A multilayered absorbent pad 35 is fixed center-symmetrically on the backing layer leaving a tacky margin 39 for fixing on a body. The absorbent pad 35 consists of a first absorbent layer 38 composed of a first absorbent fiber material, a second absorbent layer 34 consisting of a second absorbent fiber material other than the first absorbent fiber material, and a third layer 36 which is disposed between the first and second layers and distances the first and second layers. The third layer 36 consists of a netlike polyethylene foil coated with elemental silver on both sides. The first absorbent layer 38 comprises by way of absorbent material a needled fibrous nonwoven web comprising viscose fibers. The second absorbent layer 34 comprises by way of absorbent fiber material a blend of superabsorbent polyacrylate fibers and viscose fibers. The presence of superabsorbent polyacrylate fibers means that the second absorbent layer 34, when compared with the first absorbent layer 38, has a significantly larger free absorbency than the first absorbent layer. The absorbent pad further includes a wound contact layer 37 which is permeable to wound exudate, does not stick to the wound and is made of a polymer foil composed of polyethylene. The layered construction chosen keeps the wound exudate away from the wound and prevents any maceration of the wound and of the surrounding edges of the wound. The disposition of the silver-coated polymer foil between the two absorbent layers keeps the wound dressing very substantially germ-free—even in the event of a prolonged dwell time of three to five days on the wound—to prevent any recontamination of the wound with germs. The side of the wound dressing which in its use state faces the body of the patient is covered with a detachable release paper 33 for protection before use.

### Exemplary Embodiment

### I) Test Methods

### 1) Free Absorbency of Saline—Test 1

The free absorbency of physiological sodium chloride solution (0.9% by weight in strength, NaCl in water) is determined in line with EN 13726-1 (2002). The sole difference to DIN EN 13726-1 (2002) is the use of NaCl solution instead of NaCl and CaCl₂ solution. The following equation applies:

$$liquid imbibed = \frac{{M}_{2} - {M}_{1}}{{M}_{1}} [ g \text{/} g ] \qquad ( 1 )$$

M2=wet weight of sample in g
M1=dry weight of sample in g

### 2) Silver Release—Test 2

To determine the release of silver from the wound dressing, a sample of the absorbent pad (without the backing layer) of 5×5 cm is placed in a sealable Erlenmeier flask filled with 100 ml of distilled water so that the absorbent pad is completely covered by water. The sample is left in the sealed flask for 24 hours at 25° C. After 24 hours, the sample is removed from the flask, the remaining liquid is filtered and the silver content of the filtered liquid is determined using atomic absorption spectroscopy.

### II) Construction of Wound Dressing

The wound dressing has essentially the construction shown in FIG. 3, and the same terms will now be used to designate the individual constituents of the wound dressing. The wound dressing consists of a backing material composed of a water-impermeable hydroentangled fibrous nonwoven web of polyester fibers having a basis weight of 70 g/m² (Sontara 8010, DuPont). A hot-melt adhesive based on synthetic rubber has been uniformly applied to the backing material in an amount of 35 g/m². The wound dressing has a size of 10×8 cm wherein the absorbent pad (of the type P 2007/123/7/3; from Kemex—AL Almelo, Netherlands) having a size of 6×4 cm has been applied to the pressure-sensitive adhesive center-symmetrically to leave a tacky margin of 2 cm surrounding the absorbent pad on all sides. Every layer of the absorbent pad is with regard to its outer circumference equal in size to a second layer of the absorbent pad. The absorbent pad includes a wound contact layer which does not stick to wounds and to which there is laminated a first absorbent layer of a needled fibrous nonwoven web consisting of a blend of viscose fibers, polyethylene fibers and polypropylene fibers. The wound contact layer consists of a micro-porous polyethylene net having a basis weight of 32 g/m². The wound contact layer includes a multiplicity of openings or apertures for passage of liquids, these openings or apertures occupying an area of about 20% of the area of the wound contact layer. The fibrous nonwoven web of the first absorbent layer has a basis weight of 275 g/m² and a free absorbency of 1965 g/m² (measured to DIN EN 13726-1 (2002)—Test 1). Hence the free absorbency of the first absorbent layer A₁=7.1 g/g. The absorbent pad further includes an air-laid fibrous nonwoven web as second absorbent layer. The air-laid fibrous nonwoven web consists of a blend of 20% by weight (based on the air-laid fibrous nonwoven web) of superabsorbent polyacrylate fibers composed of a polyacrylate-polymethacrylate copolymer, 75% by weight of viscose fibers and 5% by weight of thermo-plastic bicomponent fibers formed from polyethylene and polypropylene. The air-laid fibrous nonwoven web has a basis weight of 85 g/m² and a free absorbency of 1800 g/m² (measured to DIN EN 13726-1 (2002)—Test 1). Hence the free absorbency of the second absorbent layer A₂=21.2 g/g. Hence the ratio of A₁ to A₂ is 1:3. Between the first and second absorbent layers there is a polyethylene polymer net coated on one side with elemental silver with the silver layer facing the first absorbent layer. The coated polymer net has a basis weight of 20 g/m² and the amount of elemental silver on the polymer net is in the range from 150 to 300 mg/m². The polymer net includes a multiplicity of openings or apertures for passage of liquids, these openings or apertures occupying an area of about 50% of the area of the polymer net. The total weight of the absorbent pad is 412 g/m², while the absorbent pad has a free absorbency of 3765 g/m² (measured to DIN EN 13726-1 (2002)—Test 1). Hence the absorbent pad has a free absorbency A_{T} of 9.1 g/g.

The wound dressing cannot be classified as an anti-microbial wound dressing, since the release rate of silver from the wound dressing amounts to merely 11.3 μg of silver per 1 g of absorbent pad over 24 hours at 25° C. in 100 ml of water with regard to silver in elemental form or in the form of salts or complexes, the amount of silver released being determined by means of atomic absorption spectroscopy. It is thus the case that the antimicrobial agent acts almost exclusively within the wound dressing. When this wound dressing is used as intended, there is accordingly no release of additional active ingredients onto or into the wound.

1-12. (canceled)

13. A wound dressing for treating a strongly or moderately exudating wound, the dressing comprising:
a backing layer; and
an absorbent pad, said absorbent pad having a first absorbent layer comprising a first absorbent material, a second absorbent layer that differs from said first layer and that comprises a second absorbent material that differs from said first material, and a third layer disposed between said first layer and said second layer, wherein said third layer comprises at least one antimicrobial agent.

14. The wound dressing of claim 13, wherein, in a use state of the wound dressing, said first absorbent layer faces and is in direct or indirect contact with the wound.

15. The wound dressing of claim 13, wherein said first absorbent material and/or said second absorbent material is a fiber material.

16. The wound dressing of claim 13, wherein said second absorbent layer has a greater free absorbency than said first absorbent layer.

17. The wound dressing of claim 13, wherein a ratio of free absorbency A₁ of said first absorbent layer to a free absorbency A₂ of said second absorbent layer is such that an A₁:A₂ ratio corresponds to a ratio in a range from 1:2 to 1:10.

18. The wound dressing of claim 13, wherein said second absorbent material comprises superabsorbent fibers.

19. The wound dressing of claim 13, wherein said third layer is a polymer net or perforated polymer foil coated with an antimicrobial agent.

20. The wound dressing of claim 13, wherein said antimicrobial agent comprises elemental silver, a silver oxide, a silver complex, a silver salt or mixtures thereof.

21. The wound dressing of claim 13, wherein said absorbent pad further comprises a wound contact layer which is in immediate contact with said first layer or said second layer.

22. The wound dressing of claim 21, wherein said wound contact layer is a polymer net or a perforated polymer foil.

23. The wound dressing of claim 21, wherein said wound contact layer and/or said first absorbent layer comprises no antimicrobial agents.

24. The wound dressing of claim 13, wherein said backing layer consists essentially of a fibrous nonwoven web or a polymer film.
